(19) United States
(12) Patent Application Publication
(10) Pub. No.: US 2009/0247850 A1
(43) Pub. Date: Oct. 1, 2009

(54) Manually Powered Oximeter

(75) Inventor: Charles E. Porges, Orinda, CA (US)

Correspondence Address: NELLCOR PURITAN BENNETT LLC;ATTN: IP LEGAL, 60 Middletown Avenue, North Haven, CT, 06473, US

(73) Assignee: Nellcor Puritan Bennett LLC, Boulder, CO (US)

(21) Appl. No.: 12/412,562

(22) Filed: Mar. 27, 2009

Related U.S. Application Data
(60) Provisional application No. 61/072,259, filed on Mar. 28, 2008.

(51) Int. Cl. A61B 5/1455

(52) U.S. Cl. 600/323

(57) ABSTRACT

Embodiments disclosed herein may include a medical device and a method for powering a medical device are disclosed. The medical device may be able to operate independent of a plug-in and a wall socket as a power source by way of a manual power source. Additionally, shock resistant components are described which may protect the medical device from damage typically encountered during manually powering and using the pulse oximeter in areas where traditional power sources such as a wall outlet are unavailable.

### RELATED APPLICATIONS

This application claims priority to U.S. Provisional Application No. 61/072,259, filed Mar. 28, 2008, and is incorporated herein by reference in its entirety.

### BACKGROUND

The present disclosure relates generally to medical devices and, more particularly, to powering medical devices.

This section is intended to introduce the reader to various aspects of art that may be related to various aspects of the present invention, which are described and/or claimed below. This discussion is believed to be helpful in providing the reader with background information to facilitate a better understanding of the various aspects of the present invention. Accordingly, it should be understood that these statements are to be read in this light, and not as admissions of prior art.

In the field of medicine, there is a need to monitor physiological characteristics of a patient. Accordingly, a wide variety of devices and techniques have been developed for monitoring the physiological characteristics of a patient. One such technique for monitoring certain physiological characteristics of a patient (e.g., blood flow characteristics) is commonly referred to as pulse oximetry. Devices which perform pulse oximetry are commonly referred to as pulse oximeters. Pulse oximeters may be used to measure physiological characteristics such as the blood-oxygen saturation of hemoglobin in arterial blood, the volume of individual blood pulsations supplying the tissue, and/or the rate of blood pulsations corresponding to each heartbeat of a patient.

Specifically, these measurements may be acquired using a non-invasive sensor that transmits electromagnetic radiation, such as light, through a patient's tissue and that photoelectrically detect the absorption and scattering of the transmitted light in such tissue. Physiological characteristics may then be calculated based upon the amount of light absorbed and scattered. More specifically, the light passed through the tissue may be selected to be of one or more wavelengths that may be absorbed and scattered by the blood in an amount correlative to the amount of blood constituent present in the tissue. The measured amount of light absorbed and scattered may then be used to estimate the amount of blood constituent in the tissue using various algorithms.

Because of the particular physiological parameters that pulse oximeters are capable of determining, the use of pulse oximeters has become important in places besides hospitals. Traditional pulse oximeters obtain power by plugging into a wall socket. However, pulse oximeters may be used to monitor and treat patients outside of a hospital setting, such as in developing nations where constant and regular sources of electricity may be difficult to obtain. This lack of a constant and regular source of electricity renders traditional plug-in pulse oximeters at a disadvantage. While pulse oximeters powered by replaceable batteries can overcome this problem, there still exists a problem that the batteries in such pulse oximeters regularly die and need to be replaced. When this occurs in situations where replacement batteries are not readily available, these pulse oximeters become similarly disadvantaged as the traditional plug-in pulse oximeters.

Additionally, current pulse oximeters typically are not rugged enough to withstand use outside of a hospital setting. The pulse oximeters designed for use today are typically intended for use in a hospital where there is very little shock that the pulse oximeter must endure. Thus, current pulse oximeters have an added problem for use in developing nations in that they typically cannot handle the rough usage that may occur in areas outside of a hospital setting.

### SUMMARY

Certain aspects commensurate in scope with the original claims are set forth below. It should be understood that these aspects are presented merely to provide the reader with a brief summary of certain embodiment and that these aspects are not intended to limit the scope of the claims. Indeed, the disclosure and claims may encompass a variety of aspects that may not be set forth below.

In accordance an embodiment there is provided a manually powered pulse oximeter that includes a manual power source. The manual power source may include a manual generator and a power storage device. The manual power source may be capable of powering the pulse oximeter without an external source of power. The manually powered pulse oximeter may also be shock resistant and capable of withstanding being shaken or dropped without damage to the internal components.

### BRIEF DESCRIPTION OF THE DRAWINGS

Advantages of the disclosure may become apparent upon reading the following detailed description and upon reference to the drawings in which:

FIG. 1 illustrates a perspective view of a pulse oximeter in accordance with an embodiment;

FIG. 1A illustrates a perspective view of a sensor in accordance with the embodiment pulse oximeter illustrated in FIG. 1;

FIG. 2 illustrates a hand held pulse oximeter in accordance with an embodiment;

FIG. 3 illustrates a hand held pulse oximeter having a remote sensor in accordance with an embodiment;

FIG. 4 illustrates a simplified block diagram of a pulse oximeter having an manual power source in accordance with an embodiment;

FIG. 5 illustrates an embodiment of a simplified block diagram of the manual power source in FIG. 4;

FIG. 6 illustrates a first manual generator in accordance with an embodiment of the manual power source of FIG. 4; and

FIG. 7 illustrates a second manual generator in accordance with an embodiment of the manual power source of FIG. 4.

### DETAILED DESCRIPTION

Various embodiments will be described below. In an effort to provide a concise description of these embodiments, not all features of an actual implementation are described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

Traditional pulse oximeters may use a wall socket as a power source and charger for batteries, and) thus, are ill-suited to treat patients outside of a hospital setting in such places as developing nations where constant and regular sources of electricity may be difficult to obtain. Additionally, current pulse oximeters typically are not rugged enough to withstand use outside of a hospital setting. To address these limitations, the present disclosure details the use of a manual power source used to power a pulse oximeter. Moreover, shock resistant components are described to protect the manually powered pulse oximeter from damage typically encountered during manually powering and using the pulse oximeter.

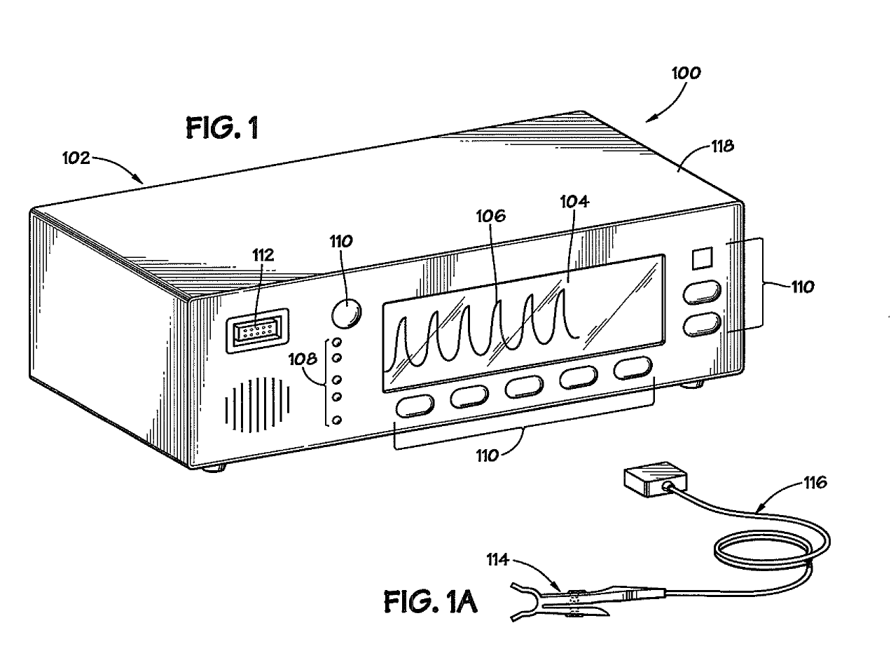

Turning to FIG. 1, a perspective view of a medical device is illustrated in accordance with an embodiment. The medical device may be a manually powered pulse oximeter 100 that includes a manual power source (not shown). The manually powered pulse oximeter may include a monitor 102. The monitor 102 may be configured to display calculated parameters on a display 104. As illustrated in FIG. 1, the display 104 may be integrated into the monitor 102. However, the monitor 102 may be configured to provide data via a port to a display (not shown) that is not integrated with the monitor 102. The display 104 may be configured to display computed physiological data including, for example, an oxygen saturation percentage, a pulse rate, and/or a plethysmographic waveform 106. As is known in the art, the oxygen saturation percentage may be a functional arterial hemoglobin oxygen saturation measurement in units of percentage SpO₂, while the pulse rate may indicate a patient's pulse rate in beats per minute. The monitor 102 may also display information related to alarms, monitor settings, and/or signal quality via indicator lights 108.

To facilitate user input, the monitor 102 may include a plurality of control inputs 110. The control inputs 110 may include fixed function keys, programmable function keys, and soft keys. Specifically, the control inputs 110 may correspond to soft key icons in the display 104. Pressing control inputs 110 associated with, or adjacent to, an icon in the display may select a corresponding option.

The monitor 102 may also include a sensor port 112. The sensor port 112 may allow for connection to an external sensor. FIG. 1A illustrates a sensor 114 that may be used with the monitor 102. The sensor 114 may be communicatively coupled to the monitor 102 via a cable 116 which connects to the sensor port 112. The sensor 114 may be of a disposable or a non-disposable type. Furthermore, the sensor 114 may obtain readings from a patient, which can be used by the monitor to calculate certain physiological characteristics such as the blood-oxygen saturation of hemoglobin in arterial blood, the volume of individual blood pulsations supplying the tissue, and/or the rate of blood pulsations corresponding to each heartbeat of a patient. The sensor 114 and the monitor 102 may combine to form the pulse oximeter 100.

The monitor 102 may also include a casing 118. The casing 118 may be made of shock resistant material such as hard plastic or hard rubber. The casing 118 may also include an internal and/or external layer of shock absorbing material such as foam or other types of insulating material. The combination of the shock resistant and shock absorbent materials used for the casing 118 ruggedizes the manually powered pulse oximeter 100, so that the manually powered pulse oximeter 100 may be shaken vigorously or dropped without damage.

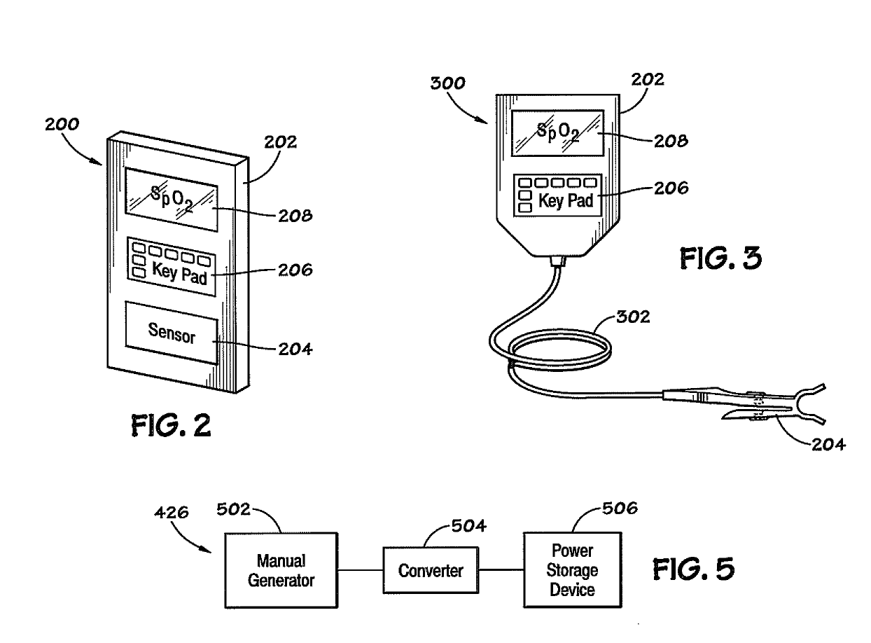

The manually powered pulse oximeter 100 may of a standard size. However, it may be beneficial to incorporate aspects of the ruggedized manually powered pulse oximeter 100 into a more portable or hand-held medical device, such as the manually powered pulse oximeter 200 illustrated in FIG. 2. The casing 202 of the portable manually powered pulse oximeter 200 may be designed to generally fit within the palm of a user's hand, making it easy to carry and convenient to use. For example, the pulse oximeter 10 may be ½ in.×1 in.×2 in. and weigh approximately 0.1 lbs. As such, a user, such as a caregiver or a patient, may carry it around in a pocket or a small bag for easy use outside of a hospital or traditional health care environment. The casing 202 may be made of shock resistant material such as hard plastic or hard rubber, and may also include an internal and/or external layer of shock absorbing material such as foam or other types of insulating material. These materials aid in ruggedizing the portable manually powered pulse oximeter 200, so that the portable manually powered pulse oximeter 200 may be shaken vigorously or dropped without damage.

In an embodiment, the portable manually powered pulse oximeter 200 may include a sensor 204, a keypad 206, and a display 208. The sensor 204 may be configured to allow the user to place a finger on the sensor pad or, alternatively, to place the sensor on a forehead. The keypad 206 may be capable of allowing a user to interface with the portable manually powered pulse oximeter 200. For example, the keypad 206 may be configured to allow a user to select a particular mode of operation. In an embodiment (not shown), the keypad 206 may not be provided. The display 208 may be oriented relative to the sensor 204 to facilitate a user reading the display 208. The display 208 may also allow a user to read the various measured parameters of the pulse oximeter, such as oxygen saturation level and/or pulse rate.

FIG. 3 illustrates an embodiment of a portable or hand-held medical device. The medical device may be a portable manually powered pulse oximeter 300 similar to the portable manually powered pulse oximeter 200 described above. The portable manually powered pulse oximeter 300 may include a casing 202, a sensor 204, a keypad 206, and a display 208, which function as described above. However, the sensor 204 is not included in the physical structure of portable manually powered pulse oximeter 300, but instead is coupled to casing 202 via a cable 302. This configuration allows for the sensor 202 and the cable 302 to be removable from the portable manually powered pulse oximeter 300. In this manner, the sensor 202 and cable 302 may be interchangeable with other components, and alternatively, may be disposable. Alternatively, another embodiment similar to this configuration allows for removal of the cable 302 altogether. In this embodiment, the sensor 204 may transmit information wirelessly to the portable manually powered pulse oximeter 300.

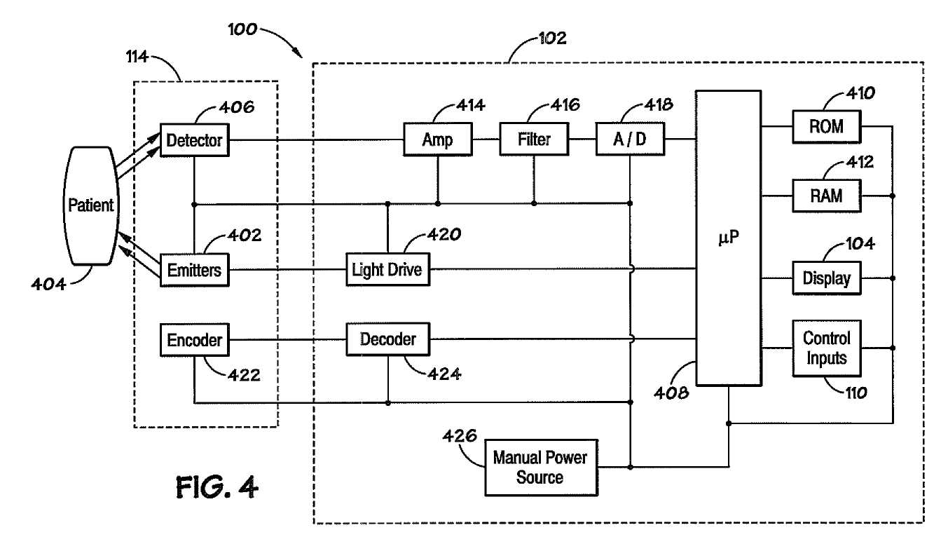

Although the size and location of the sensors 114 and 202 differ with respect to the three pulse oximeters 100, 200, and 300 described above, the internal circuitry may be similar amongst the three. FIG. 4 illustrates a simplified block diagram of an embodiment of the manually powered pulse oximeter 100, however, the block diagram may equally apply to the portable manually powered pulse oximeters 200 and 300. The manually powered pulse oximeter 100 may include a sensor 114 having an emitter 402 configured to transmit electromagnetic radiation, i.e., light, into the tissue of a patient 404. The emitter 402 may include a plurality of LEDs operating at discrete wavelengths, such as in the red and infrared portions of the electromagnetic radiation spectrum for example. Alternatively, the emitter 402 may be a broad spectrum emitter.

The sensor 114 may also include a detector 406. The detector 406 may be a photoelectric detector which may detect the scattered and/or reflected light from the patient 404. Based on the detected light, the detector 406 may generate an electrical signal, e.g. current, at a level corresponding to the detected light. The sensor 114 may direct the electrical signal to the monitor 102, where the electrical signal may be used for processing and calculation of physiological parameters of the patient 404.

In this embodiment, the monitor 102 may be a pulse oximeter, such as those available from Nellcor Puritan Bennett L.L.C. Further, the monitor 102 may include an amplifier 414 and a filter 416 for amplifying and filtering the electrical signals from the sensor 114 before digitizing the electrical signals in the analog-to-digital converter 418. Once digitized, the signals may be used to calculate the physiological parameters of the patient 404. The monitor 102 may also include one or more processors 408 configured to calculate physiological parameters based on the digitized signals from the analog-to-digital converter 418 and further using algorithms programmed into the monitor 102. The processors 408 may be connected to other component parts of the monitor 102, such as one or more read only memories (ROM) 410, one or more random access memories (RAM) 412, the display 104, and the control inputs 110. The ROM 410 and the RAM 412 may be used in conjunction, or independently, to store the algorithms used by the processors in computing physiological parameters. The ROM 410 and the RAM 412 may also be used in conjunction, or independently, to store the values detected by the detector 406 for use in the calculation of the aforementioned algorithms. The control inputs 110, as described above, may allow a user to interface with the monitor 102.

Further, the monitor 102 may include a light drive unit 420. Light drive unit 420 may be used to control timing of the emitter 402. An encoder 422 and decoder 424 may be used to calibrate the monitor 102 to the actual wavelengths being used by the emitter 402. The encoder 422 may be a resistor, for example, whose value corresponds to the actual wavelengths and to coefficients used in algorithms for computing the physiological parameters. Alternatively, the encoder 422 may be a memory device, such as an EPROM, that stores wavelength information and/or the corresponding coefficients. For example, the encoder 442 may be a memory device such as those found in OxiMax® sensors available from Nelicor Puritan Bennett L.L.C. The encoder 442 may be communicatively coupled to the monitor 102 in order to communicate wavelength information to the decoder 424. The decoder 424 may receive and decode the wavelength information from the encoder 422. Once decoded, the information may be transmitted to the processors 408 for utilization in calculation of the physiological parameters of the patient 404.

The monitor 102 may also include a manual power source 426. The manual power source 426 may be used to transmit power to the components located in the monitor 102 and/or the sensor 114. The manual power source 426 may harness kinetic energy derived from a user and convert the kinetic energy into usable power, for example electricity, that the components in monitor 102 and sensor 114 use to function.

Examples of the components utilized in the manual power source 426 to harness and convert the kinetic energy provided by a user are illustrated in FIG. 5, which illustrates a simplified block diagram of a manual power source 426. The manual power source 426 may include a manual generator 502. The manual generator 502 converts kinetic energy into usable power. The manual generator 502 may be used to generate an alternating current through inductance. For example, kinetic energy input by the user may be translated into alternating current through the inductive characteristics and arrangement of the components of the manual generator 502. This generated current may then be transmitted to the converter 504. The converter 504 rectifies the alternating current transmitted from the manual generator 502 into direct current. The converter 504 may be a full wave rectifier made up of, for example, diodes. The rectification of the electricity by the converter 504 may also include smoothing the output of the converter 504. A filter, such as a reservoir capacitor, may be used to smooth the output of the converter 504. The smoothed direct current may then be transmitted a power storage device 506. The power storage device 506 stores the generated and converted power for use by the components of monitor 102 and sensor 114. In one embodiment, power storage device 506 may include one or more rechargeable batteries. In another embodiment, the power storage device 506 may include one or more capacitors.

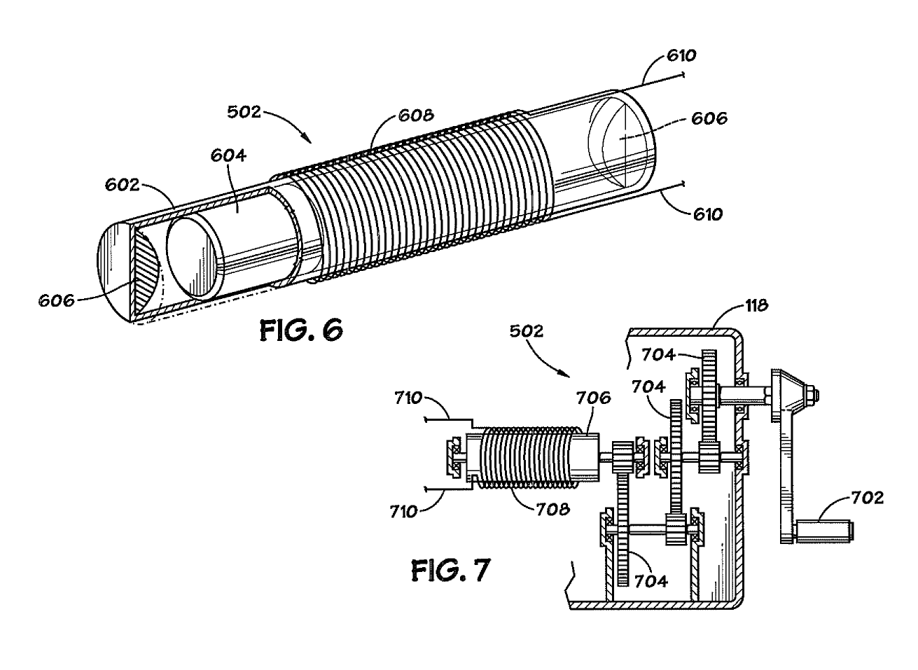

The manual generator 502 may include a variety of kinetic energy generation systems. One such system is illustrated in FIG. 6. The manual generator 502 includes a case 602, a magnet 604, one or more buffers 606, a coil 608, and one or more leads 610. The case 602 may be composed of plastic or any other non-conducting material. The case 602 may enclose the magnet 604 and the buffers 606. The case 602 may also be sized to allow lateral movement of magnet 604. In one embodiment, the case 602 is cylindrical in shape.

The magnet 604 may be sized to fit within the case 602 and move laterally within the case 602. The magnet 604 may be a permanent magnet. The magnet 604 may be capable of sliding from one end of the case 602 to the other in response to an input of kinetic energy. In one embodiment, the kinetic energy may include a user shaking the manual generator 502. The movement of the magnet 604 through the case 602 causes the magnet to pass through the coil 608. The coil 608 may be made up of a conductive substance and may be wrapped around the case 602. In one embodiment, the coil 608 may be made from coiled aluminum. In another embodiment, the coil may be made from coiled copper wire. The copper wire may be covered by thin insulation.

As the magnet 604 passes through the coil 608, electricity is generated via electromagnetic induction. This electricity may then be transmitted via the leads 610 to the converter 504. The converter 504 may include a rectifier circuit, as described above. Additionally, the converter 504 may include a transformer (not pictured) or a phase converter (not pictured). The leads 610 may be made from a conductive material such as metal wire. Additionally, the leads 610 may include a single wire, two wires, or three wires, allowing the leads 610 to conduct one, two, or three phase power.

The magnet 604 also may contact buffers 606 as it passes through the case 602. The buffers 606 may be made of elastic material such as rubber. In another embodiment, the buffers 606 may be springs. The buffers 606 at to help conserve the kinetic energy being focused into the sliding magnet 604 by redirecting the magnet 604 back through the case 602 when the buffer 606 is contacted by the magnet 604. In this manner, the buffers 606 aid in the conversion of kinetic energy into usable electricity.

Another embodiment for the manual generator 502 is illustrated in FIG. 7. The manual generator 502 may include a handle 702. The handle 702 may be rotatable about an axis. The handle 702 may also be foldable (not shown) into the casing 118 for ease of storage when not in use. The handle 702 may be connected to a gear train 704. As a user cranks the handle in a circular direction, the gear train 704 acts to transfer the rotational torque from the handle 702 to a magnet 706. In one embodiment, the gear train 704 is set to create increased rotations of the magnet 706 relative to the handle 702. The magnet 706 may rotate inside of a coil 708. The rotational motion of the magnet 706 inside the coil 708 induces an electrical current in the coil 708 which may be transmitted via conductive leads 710 to the converter 504. Converter 504 may include a rectifier circuit, a transformer, or a phase converter. Moreover, the leads 710, which may be made from a conductive material, may include a single wire, two wires, or three wires, allowing the leads 710 to conduct one, two, or three phase power. Through the use of these leads 710, the manual generator 502 may convert inputted kinetic energy, here the cranking of a handle, into electricity useable by the pulse oximeter 100. The manual power source may also work similarly to watches which do not need to b wound, or powered with a battery.

Various embodiments have been shown by way of example in the drawings and have been described in detail herein. However, it should be understood that the claims are not intended to be limited to the particular forms disclosed. Rather, the claims are to cover all modifications, equivalents, and alternatives falling within their spirit and scope.

What is claimed is:

1. A medical device comprising:
a monitor adapted obtain a physiologic signal from a patient;
a processor adapted to calculate physiological characteristics of the patient based at least in part on the physiologic signal; and
a manual power source adapted to power the monitor and the processor.

2. The medical device of claim 1, wherein the manual power source comprises:
a manual generator adapted to convert kinetic energy into electricity;
a converter adapted to rectify the electricity; and
a power storage device adapted to store the rectified electricity.

3. The medical device of claim 2, wherein the manual generator comprises:
a magnet;
a case in which the magnet is disposed while allowing for lateral movement of the magnet; and
a conductor coiled around the case.

4. The medical device of claim 2, wherein the manual generator comprises:
a magnet located inside a coiled conductor;
a gear train coupled to the magnet and adapted to rotate the magnet; and
a handle coupled to the gear train and adapted to transfer rotational torque to the magnet via the gear train.

5. The medical device of claim 2, wherein a power storage device comprises one or more capacitors.

6. The medical device of claim 2, wherein a power storage device comprises one or more rechargeable batteries.

7. The medical device of claim 1, comprising a shock resistant casing.

8. The medical device of claim 1, wherein the medical device comprises a pulse oximeter.

9. The medical device of claim 1, wherein the monitor is sized to generally fit within the palm of a user's hand.

10. The medical device of claim 1, comprising a sensor adapted to emit electromagnetic radiation into a tissue sample of the patient and detect the scattered and reflected light from the tissue sample.

11. The medical device of claim 10, wherein the sensor is adapted to generate the physiologic signal corresponding to the scattered and reflected light detected and to direct the physiologic signal to the monitor.

12. The medical device of claim 11, wherein the manual power source is capable of powering the sensor.

13. A method of powering a medical device comprising:
inputting kinetic energy into a manual generator in the medical device;
converting the kinetic energy into electricity; and
storing the electricity in the medical device for use by the medical device.

14. The method of claim 13, wherein inputting kinetic energy comprises shaking the medical device.

15. The method of claim 13, wherein converting the kinetic energy into electricity comprises moving a magnet through a coiled conductor in response to the shaking of the medical device.

16. The method of claim 13, wherein inputting kinetic energy comprises cranking a handle attached to the medical device.

17. The method of claim 16, wherein converting the kinetic energy into electricity comprises transferring rotational torque of the handle to a magnet via a gear train.

18. The method of claim 13, comprising rectifying the electricity.

19. A medical device comprising:
a storage device capable of being charged by induced current; and
a monitor adapted to obtain a physiologic signal from a patient, wherein the monitor is powered by the storage device.

20. The medical device of claim 18, wherein the induced current is generated by kinetic energy inputted into the medical device.
